(19) United States
(12) Patent Application Publication
(10) Pub. No.: US 2010/0228201 A1
(43) Pub. Date: Sep. 9, 2010

(54) Reusable Intravenos Flow Regulator

(76) Inventor: Chianhua Tan, Bulacan (PH)

Correspondence Address: Chianhua Tan, Lot 18 Blk 22 Ipil-Ipip, Street Ph 1, Marilao, 3019, PH

(21) Appl. No.: 12/308,612

(22) PCT Filed: Jun. 1, 2007
(86) PCT No.: PCT/PH2007/000009

§ 371 Date: May 19, 2010

(30) Foreign Application Priority Data
Jun. 6, 2006 (PH) 1-2006-000292

(51) Int. Cl. A61M 39/00

(52) U.S. Cl. 604/250

(57) ABSTRACT

This invention relates to an intravenous flow regulator that can be reused to address the environmental problem caused by disposable type regulators and to bHng down medical expenses, regardless of the degree of significance. The regulator is characterized by a unit generally consisting of a five-sided body and a regulating screw. The five-sided sided-body has a threaded hole for the regulating screw and a canal with a generally L-shaped section where the intravenous tube is to be accommodated. The threaded hole is bored through the canal.

This invention generally relates to regulators but more particularly to a reusable intravenous flow regulator.

### BACKGROUND OF THE INVENTION

Presently available intravenous flow regulator is defined by a plastic piece or body having a channel or groove where a regulating eccentric wheel is accommodated. The wheel is to regulate the flow of intravenous liquid by rolling on the intravenous tube passing through the intravenous flow regulator. The present intravenous flow regulator is disposable type, thereby making it additional item for the problematic volume of garbage or trash affecting the environment.

### SUMMARY OF THE INVENTION

It is one object of the present invention to provide a reusable intravenous flow regulator that is reusable for a number of usages, thereby eliminating an additional item for garbage collection.

Another object of the present invention in to bring down medical expenses by providing an item which can be reused by the medical personnel or the patients.

These, and other objects and advantages of the present invention for a reusable intravenous flow regulator will be fully understood and readily appreciated after reading the following detailed description made in conjunction with the accompanying drawings, wherein:

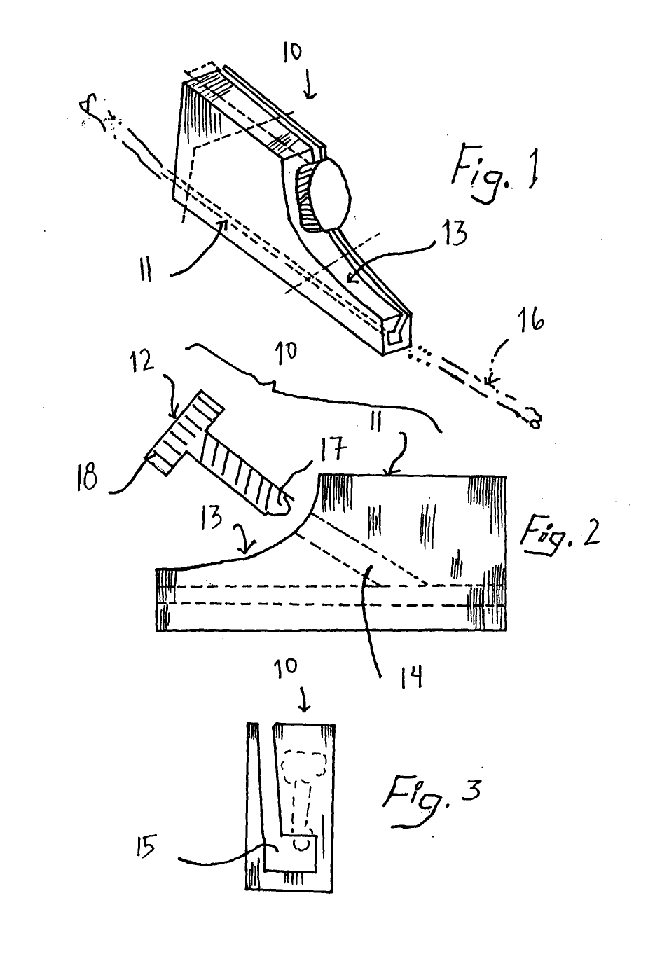

FIG. 1. is a front perspective view of the present invention for a reusable intravenous flow regulator,

FIG. 2. is a side view of the same regulator shown in FIG. 1, with the flow regulating screw being exploded or unscrewed therefrom; and

FIG. 3. is a rear end view of the regulator.

Referring now to the drawings, there is shown the present novel reusable intravenous flow regulator designated in its entirety by reference numeral 10 and generally consisting of a monolithic generally five—sided body 11 and a flow regulating screw 12.

As best shown in FIG. 2 in conjunction with FIG. 3 the monolithic generally five-sided body 11 has a slanting or bevelled side 13 being provided with a perpendicularly disposed threaded hole 14 adapted to accommodate the flow regulating screw 12, and a generally L-shaped canal or cavity 15 being provided to accommodate the intravenous tube 16. Obviously, the threaded hole 14 bores or extends through the L-shaped canal 15, to thereby allow the lower tip 17 of the regulating screw 12 to press down on the intravenous tube 16 being a accommodated with in the lower portion of the canal 15.

The flow regulating screw 12 is defined by threaded body, preferably, having a turning head 18 with abrasive or rough surface to facilitate turning,

The present reusable intravenous flow regulator 10 is preferably made of light materials such as but not limited to, light plastic material, inherently light aluminum material and composite,

I claim:

1. A reusable intravenous flow regulator comprising a generally at least five-sided body and having a generally L-shaped canal adapted to accommodate an intravenous tube, and a regulating screw being screwably accommodated in a threaded hole disposed perpendicularly at the slanting side of the five-sided body, the threaded hole extending through the L-shaped canal to thereby allow the regulating screw to press on the intravenous tube in the canal.
